(19) United States
(12) Patent Application Publication
(10) Pub. No.: US 2026/0368970 A1
(43) Pub. Date: Nov. 19, 2026

(54) RADIOGRAPHIC IMAGE PROCESSING APPARATUS, GENERATION METHOD, AND RADIOGRAPHIC IMAGING SYSTEM

(71) Applicant: Konica Minolta, Inc., Tokyo (JP)

(72) Inventors: Naoki HAYASHI, Tokyo (JP); Ryohei ITO, Tokyo (JP)

(21) Appl. No.: 19/674,361

(22) Filed: May 12, 2026

(30) Foreign Application Priority Data
May 14, 2025 (JP) 2025-081194

(51) Int. Cl. G06T 11/60; A61B 6/46

(52) U.S. Cl. CPC G06T 11/60; A61B 6/463; G06T 2210/41

(57) ABSTRACT

Provided is a radiographic image processing apparatus including a hardware processor that displays, on a display medium during imaging, a first dynamic image. The hardware processor recognizes change processing on the first dynamic image, generates, based on the first dynamic image, a second dynamic image, and applies the change processing to the second dynamic image.

### CROSS REFERENCE TO RELATED APPLICATIONS

The entire disclosure of Japanese Patent Application No. 2025-081194, filed on May 14, 2025, is incorporated herein by reference in its entirety.

### BACKGROUND

### 1. Technological Field

The present invention relates to a radiographic image processing apparatus, a generation method, and a radiographic imaging system.

### 2. Description of the Related Art

In recent years, dynamic radiographic imaging systems that can be introduced without significant equipment changes in the general imaging environment have begun to be widely adopted. Among dynamic radiographic imaging systems, with the widespread use of medical carts for dynamic radiography, the use environments have expanded to surgery, emergency care, ward rounds, and the like, and needs for applications of dynamic radiographic imaging have become diversified.

In dynamic radiographic imaging in the related art, by performing analysis processing on captured dynamic images at a dynamic analysis apparatus, it is possible to extract morphological changes associated with temporal changes, such as ventilation and blood flow, which cannot be visually recognized in still images. Such a post-imaging dynamic image (main image) needs to be outputted and saved as an image suitable for dynamic analysis, and is therefore saved with various corrections applied to the captured dynamic image. Thereafter, the main image is displayed on a display medium.

In addition, to ensure immediacy in checking a radiographic dynamic image, for example, Japanese Patent Application Laid-Open No. 2022-92758 discloses a configuration in which, at a timing before a main image is displayed on a display medium, a preview image (still image) subjected to processing simpler than that performed on the main image is displayed on the display medium.

### SUMMARY

Incidentally, with the diversification of the above-described needs, applications of live display (display of radiographic dynamic images during imaging), such as fluoroscopy, are also demanded in dynamic radiographic imaging. Specifically, in dynamic radiographic imaging, a use method composed mainly of live display, such as swallowing, confirmation of catheter insertion, or the like, is also demanded in the market.

In a radiographic dynamic image during live display, it may become necessary to perform some kind of change processing during imaging. The some kind of change processing includes, for example, relatively simple processing, such as changing an orientation of an image from that in an initial display state, and relatively detailed processing, such as changing gradation or the like from that in the initial display state. For example, during live display, the user may actually perform change processing, or may consider that change processing is required from that time point. In addition, there is also a case where even when the user is not viewing a live display, it may be possible to determine, based on the imaging purpose and/or imaging content, that change processing is required starting from a predetermined time point.

However, in a dynamic radiographic imaging system in the related art, subsequent analysis processing is anticipated, and thus, every frame image of the main image is saved in an initial display state for live display. In addition, a fluoroscopic imaging system has no subsequent analysis processing, and thus, there is no concept of redisplaying what was displayed live. That is, in the related art, there is no concept of reproducing a live display. That is, in the dynamic radiographic imaging system in the related art, even when change processing is performed during live display, the change processing is not applied to the main image. For this reason, in a case where it is desired to reproduce a live display, the user needs to check the main image and perform the same change processing as in the live display, which was inconvenient.

An object of the present invention is to provide a radiographic image processing apparatus, a generation method, and a radiographic imaging system each capable of improving convenience in a radiographic imaging system capable of live display.

In order to achieve at least one of the above-described objects, a radiographic image processing apparatus reflecting an aspect of the present invention comprises a hardware processor that displays, on a display medium during imaging by a radiographic imaging apparatus, a first dynamic image of a subject irradiated with radiation.

The hardware processor
recognizes change processing on at least one of a plurality of frame images included in the first dynamic image,
generates, based on the first dynamic image, a second dynamic image to be displayed on the display medium after the imaging by the radiographic imaging apparatus,
and applies the change processing to the second dynamic image.

In order to achieve at least one of the above-described objects, a generation method reflecting an aspect of the present invention is a generation method in a radiographic image processing apparatus. The generation method comprises:
displaying, on a display medium during imaging by a radiographic imaging apparatus, a first dynamic image of a subject irradiated with radiation;
recognizing change processing on at least one of a plurality of frame images included in the first dynamic image; and
generating, based on the first dynamic image, a second dynamic image to be displayed on the display medium after the imaging by the radiographic imaging apparatus, where
generating the second dynamic image includes applying the change processing to the second dynamic image.

In order to achieve at least one of the above-described objects, a radiographic imaging system according to an aspect of the present invention comprises:
a radiation irradiation apparatus capable of capturing a radiographic dynamic image; and the radiographic image processing described above.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a radiographic imaging system according to an embodiment of the present invention;

FIG. 2 is a block diagram illustrating the configuration of a main part of the radiography control apparatus according to the present embodiment;

FIG. 3 is a schematic diagram illustrating pulsed irradiation in a normal imaging mode;

FIG. 4 is a schematic diagram illustrating pulsed irradiation in an intermittent imaging mode;

FIG. 5 is a block diagram illustrating the configuration of an imaging control section;

FIG. 6 is a diagram provided for describing a flow of processing until a second dynamic image is generated from a first dynamic image;

FIG. 7 is a diagram illustrating a display example of a display section;

FIG. 8 is a flowchart illustrating exemplary operations of processing performed by the imaging control section; and

FIG. 9 is a diagram provided for describing a flow of processing until a second dynamic image is generated from a first dynamic image according to a variation.

### DETAILED DESCRIPTION OF EMBODIMENTS

Hereinafter, one or more embodiments of the present invention will be described with reference to the drawings. However, the scope of the invention is not limited to the disclosed embodiments.

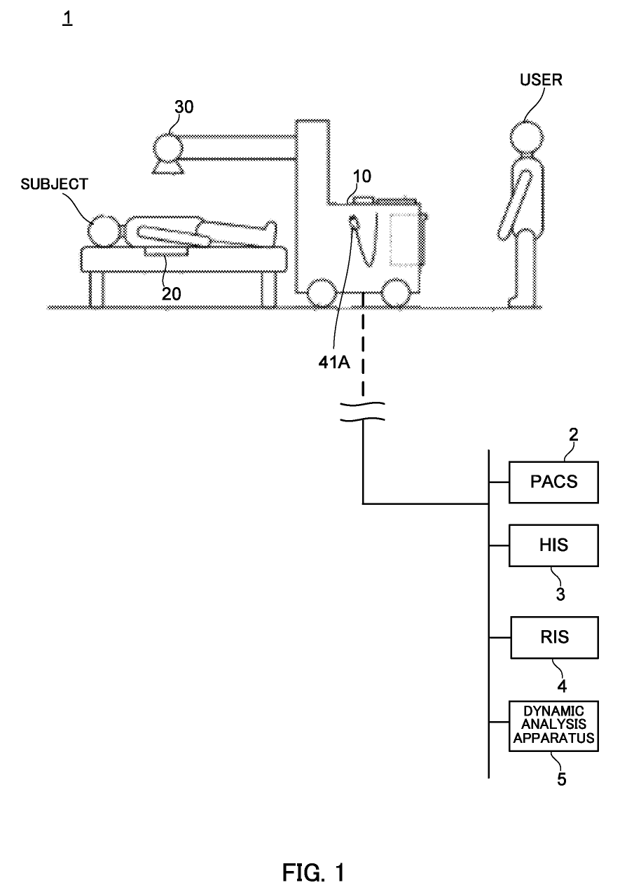

Hereinafter, an embodiment of the present invention will be described in detail with reference to the accompanying drawings. FIG. 1 is a diagram illustrating a radiographic imaging system according to an embodiment of the present invention.

As illustrated in FIG. 1, the radiographic imaging system 1 according to the present embodiment is capable of capturing still images and dynamic images, and is a visiting system for visiting a subject who has difficulty in movement, and capturing a radiographic image. Note that, the radiographic imaging system 1 is applicable to, for example, a hospital room, an intensive care unit (ICU), an operating room, and the like.

The radiographic imaging system 1 includes a main body 10, at least one flat panel detector (FPD) 20, and a radiation irradiation apparatus 30.

In addition, external systems are connected to the radiographic imaging system 1 via a communication network. Examples of the external systems include picture archiving and communication systems (PACS) 2, and hospital information systems (HIS) 3, radiology information systems (RIS) 4. In addition, a dynamic analysis apparatus 5 as an external apparatus is connected to the radiographic imaging system 1 via the communication network.

In the communication network including the radiographic imaging system 1, the PACS 2, the HIS 3, the RIS 4, and the dynamic analysis apparatus 5, information is transmitted and received, for example, in accordance with a digital image and communications in medicine (DICOM) standard.

The FPD 20 is a portable radiation detector that is compatible with capturing of still images and dynamic images. The FPD 20 includes, for example, radiation detection elements two-dimensionally arranged on a glass substrate. The radiation detection element is constituted by a semiconductor image sensor such as a photodiode. The radiation detection element detects radiation, which has been irradiated from the radiation irradiation apparatus 30 (radiation source) and has passed through at least a subject, according to the intensity thereof, converts the detected radiation into an electrical signal, and accumulates the electrical signal. For example, a switching section such as a thin film transistor (TFT) is connected to each radiation detection element. The switching section controls the accumulation and reading of electrical signals, and image data is acquired.

Note that, the FPD 20 may be of an indirect conversion type which converts radiation into an electrical signal by a photoelectric conversion element via a scintillator, or may be of a direct conversion type which directly converts radiation into an electrical signal.

In addition, the FPD 20 is connected to the main body 10 of the radiographic imaging system 1 via the communication network, and is configured to perform wired communication or wireless communication with the main body. Specifically, the FPD 20 is configured to receive various control signals from the main body 10 and transmit generated image data to the main body 10 via a communication cable.

In addition, the main body 10 is provided with a housing section (not illustrated) capable of housing the FPD 20. The main body 10 is conveyed to a hospital room in a state in which the FPD 20 is housed in the housing section.

The radiation irradiation apparatus 30 includes a synchronization signal output section (not illustrated), a generator (not illustrated), a radiation source (not illustrated), and the like.

The synchronization signal output section outputs a pulsed synchronization signal to each of the generator and the FPD 20 in response to an operation of an exposure switch 41A, which will be described later. When capturing still images, a synchronization signal is transmitted only once per still image, while when capturing dynamic images, a synchronization signal of the same cycle is repeatedly transmitted a plurality of times.

The generator is configured to be capable of applying, to the radiation source, a voltage corresponding to radiation irradiation conditions set in advance (a tube voltage, a tube current, an irradiation time, and the like) each time a synchronization signal is inputted from the synchronization signal output section.

The radiation source (tube) includes a rotating anode, a filament, and the like. The rotating anode is constituted by, in addition to a target, a rotor or an anode rotor that rotates the anode, an anode shaft, a bearing (not illustrated), and the like. The anode rotor rotates the target at a high speed by the principle of an induction motor. The filament is provided on a cathode sleeve disposed at a position at which the filament faces the target. When a voltage is applied from the generator, the filament emits an electron beam corresponding to the voltage toward the rotating anode, and the rotating anode (anode rotor) generates a dose of radiation corresponding to the intensity of the electron beam.

In addition, the radiation source is rotated by a rotation shaft extending in the horizontal direction, and can be switched to a state in which an irradiation port of radiation is directed in the horizontal direction (a state in which imaging in a standing position is performed) or a state in which the irradiation port is directed in the vertical direction (a state in which imaging in a lying position is performed).

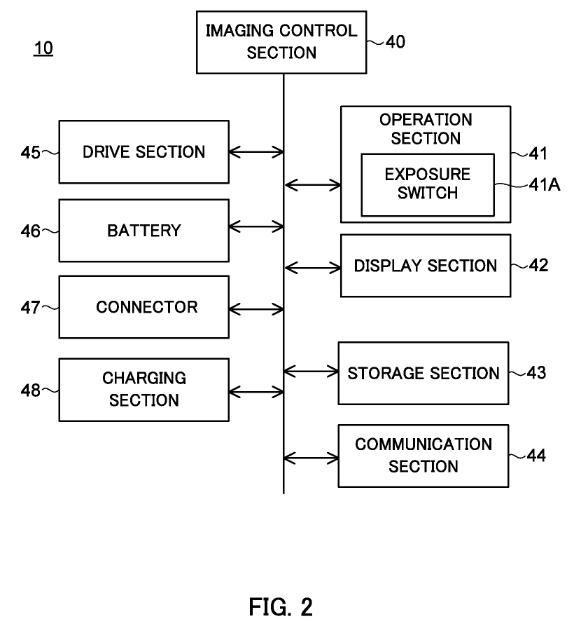

In addition, the main body 10 has a function as a console (radiographic imaging control apparatus). As illustrated in FIG. 2, the main body 10 includes an imaging control section 40, an operation section 41, a display section 42, a storage section 43, a communication section 44, a drive section 45, a battery 46, a connector 47, a charging section 48, and the like.

The imaging control section 40 is housed in the main body 10 and includes a central processing unit (CPU), a read only memory (ROM), a random access memory (RAM), and the like. In the imaging control section 40, the CPU reads a program corresponding to the processing content from the ROM and loads the read program into the RAM. Then, the imaging control section 40 cooperates with the loaded program to centrally control the operation of each section.

In addition, the imaging control section 40 performs control such that radiographic images are captured in an intermittent imaging mode in which dynamic images or still images can be captured by the FPD 20 a plurality of times during a single imaging period from the imaging start to the imaging end.

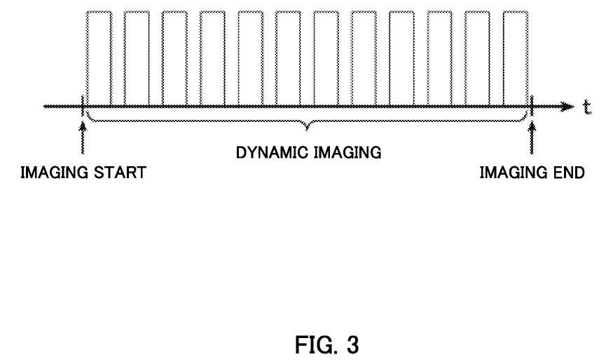

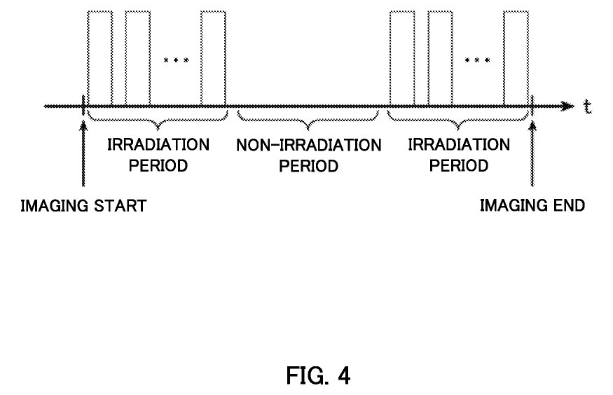

In addition, the radiographic imaging system 1 according to the present embodiment has, as imaging modes that involve dynamic images, a normal imaging mode and the intermittent imaging mode. FIG. 3 is a diagram schematically illustrating pulsed irradiation in the normal imaging mode. FIG. 4 is a diagram schematically illustrating pulsed irradiation in the intermittent imaging mode.

As illustrated in FIG. 3, the normal imaging mode is an imaging mode in which dynamic images are captured by continuously performing pulsed irradiation from a radiation source during a single imaging period from the imaging start to the imaging end. The single imaging period in the normal imaging mode is a period during which continuous pulsed irradiation for capturing dynamic images is performed, and is set based on a total dose in single imaging. In the normal imaging mode, a dynamic image including a series of a plurality of frames corresponding to the entire imaging period set in the normal imaging mode is acquired.

The intermittent imaging mode is an imaging mode in which dynamic images or still images can be captured by the FPD 20 a plurality of times during a single imaging period form the imaging start to the imaging end. Specifically, the intermittent imaging mode is an imaging mode in which an irradiation period of radiation and a non-irradiation period of radiation are alternately generated such that the irradiation period of radiation occurs at least twice within a single imaging period from the imaging start to the imaging end. Note that, the intermittent imaging mode is an imaging mode in which at least two irradiation periods are scheduled, or an imaging mode in which at least two irradiation periods can occur. For this reason, for example, in a case where a single imaging period is caused to have ended before the second irradiation period starts due to some kind of factor such as the relationship with the total dose, imaging may end after only a single irradiation period even in the intermittent imaging mode.

In addition, during imaging by the radiation irradiation apparatus 30, the imaging control section 40 causes the display section 42 to display a radiographic dynamic image during the imaging (a first radiographic dynamic image), and causes a main image (a second dynamic image) to be generated based on the first radiographic dynamic image. The generation processing of generating the second dynamic image by the imaging control section 40 will be described later.

The operation section 41 includes a touch screen or the like in which transparent electrodes are arranged in a lattice shape so as to cover the surface of the display section 42. The touch screen detects a position pressed with a finger, a touch pen, or the like, and inputs the position information as operation information to the imaging control section 40.

In addition, the operation section 41 includes an exposure switch 41A for operating the radiation irradiation apparatus 30. The exposure switch 41A is a switch for the user to instruct radiation irradiation by the radiation irradiation apparatus 30.

The display section 42 is constituted by a monitor such as a liquid crystal display (LCD) or a cathode ray tube (CRT). The display section 42 performs display in accordance with an instruction of a display signal inputted from the imaging control section 40.

The storage section 43 is constituted by a non-volatile semiconductor memory, a hard disk, or the like. The storage section 43 stores various programs executed by the imaging control section 40, parameters necessary for execution of processing by the programs, or data such as processing results.

In addition, the storage section 43 stores examination order information acquired from the RIS 4. Here, the examination order information includes information on the subject and examination information. The information on the subject includes the subject ID, name, sex, age, hospital room (ward), and the like of the subject to be examined. The examination information includes the examination ID, examination date, and imaging order of the respective pieces of imaging performed during the examination. The imaging order includes the imaging site, imaging direction, distinction between still image capturing and dynamic image capturing, and the like.

In addition, the storage section 43 is provided with a primary storage region (not illustrated) for temporarily storing the radiographic image transferred from the FPD 20. Further, the storage section 43 is provided with an image saving region (not illustrated) for saving the radiographic image transferred from the FPD 20 in association with supplementary information for a certain period of time.

The communication section 44 transmits and receives data to and from the FPD 20 by wired communication or wireless communication. In addition, the communication section 44 transmits and receives data to and from external apparatuses, such as the PACS 2, the HIS 3, the RIS 4, and the dynamic analysis apparatus 5, via the communication network. For example, the communication section 44 acquires information on an examination order inputted to an external system such as the HIS 3 or the RIS 4, information on a radiographic dynamic image captured by the radiographic imaging system 1, and information on an analysis result of a radiographic dynamic image by the dynamic analysis apparatus 5. In addition, the communication section 44 transmits a radiographic dynamic image, which has been subjected to display processing for display on a display medium, and information on an analysis result of the radiographic dynamic image or the like to the PACS 2 or the like.

The drive section 45 is a circuit that drives the tube of the radiation source of the radiation irradiation apparatus. The drive section 45 and the radiation source are connected to each other via a cable.

The battery 46 supplies electric power to each section of the main body 10, and the radiation source. The battery 46 can be charged from the outside via an AC cable.

The connector 47 is provided inside the housing section and is electrically connected to the FPD 20 housed in the housing section.

The charging section 48 charges the FPD 20, which is connected thereto via the connector 47, with the electric power supplied from the battery 46 on the basis of the control by the imaging control section 40.

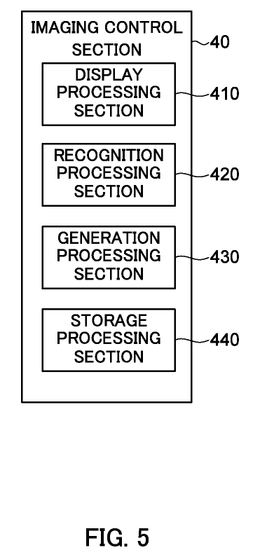

Next, the generation processing of generating the second dynamic image by the imaging control section 40 will be described. FIG. 5 is a block diagram illustrating the configuration of the imaging control section 40.

As illustrated in FIG. 5, the imaging control section 40 includes a display processing section 410, a recognition processing section 420, a generation processing section 430, and a storage processing section 440.

The display processing section 410 displays, on the display section 42 during the imaging by the radiation irradiation apparatus 30, a first dynamic image of a subject irradiated with radiation. That is, the first dynamic image is a dynamic image in which the subject in a state of having been irradiated with radiation by the radiation irradiation apparatus 30 is displayed on the display section 42 in real time (live display). In addition, the display processing section 410 may continue to display the first dynamic image on the display section 42 in a state in which the first dynamic image is can be played back even after the imaging by the radiation irradiation apparatus 30 is completed.

In the normal imaging mode, the first dynamic image is every dynamic image captured during the imaging period of the normal imaging mode. In addition, in the intermittent imaging mode, the first dynamic image is a dynamic image included in a plurality of captured images captured a plurality of times during a single imaging period from the imaging start to the imaging end in the intermittent imaging mode.

For example, in a case where dynamic images are captured three times in the intermittent imaging mode, each of the dynamic images captured three times is the first dynamic image. In addition, in a case where a still image is captured once and dynamic images are captured twice in the intermittent imaging mode, each of the dynamic images captured twice is the first dynamic image. In addition, every frame image included in a plurality of captured images including a still image(s) may constitute one first dynamic image in the intermittent imaging mode.

The recognition processing section 420 recognizes change processing on at least one of a plurality of frame images included in the first dynamic image.

The change processing is processing for changing at least one of adjustment of a region of interest, setting of an effective image region, adjustment of an image size, image rotation, image inversion, adjustment of an S/G value, image black-and-white inversion, predetermined image processing, enhancement processing, and/or grid pattern removal processing.

The predetermined image processing includes gradation processing, equalization processing, frequency enhancement processing, hybrid (H) processing, scattered radiation correction processing, changing processing of changing image processing conditions, or the like. Note that, when the predetermined image processing, the enhancement processing, or the grid pattern removal processing is applied to, for example, the first dynamic image during live display, the processing is performed by relatively simple processing. In addition, the H processing may be optimized in combination with the equalization processing, the frequency enhancement processing, smoothing processing, and the like.

Specifically, the recognition processing section 420 recognizes, as the change processing described above, processing performed on the first dynamic image displayed on the display section 42 based on operation information on the operation of the user.

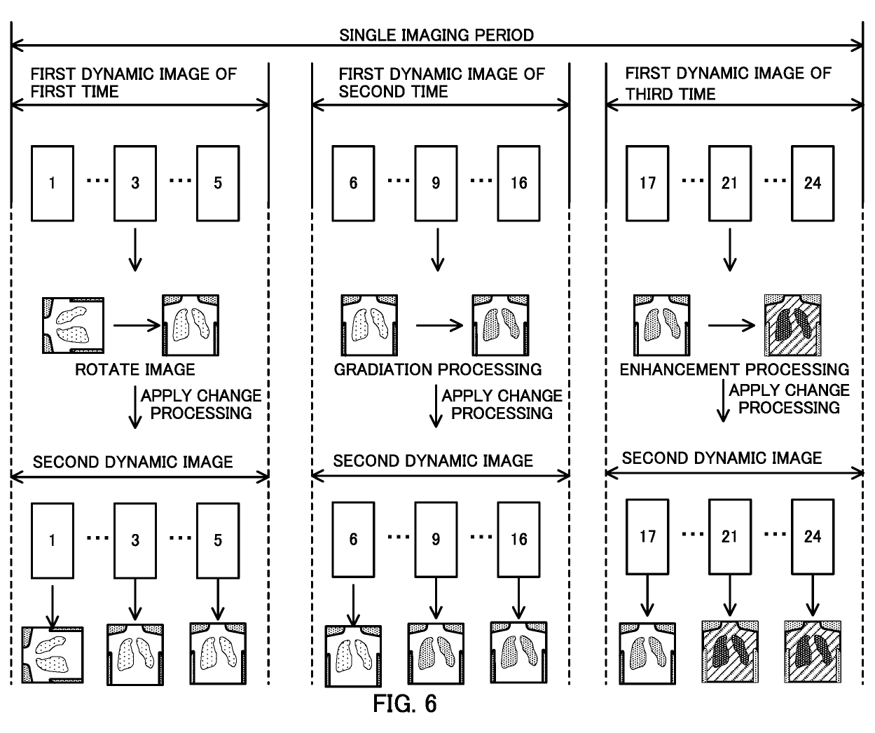

For example, it is assumed that a total of 24 frame images have been captured in a single imaging period in the intermittent imaging mode, where dynamic images have been captured three times in the single imaging period. In the example illustrated in FIG. 6, the frame images are depicted with rectangles, and the numbers in the frame images indicate the order of the frame images in a time series. In addition, in the example illustrated in FIG. 6, the first to fifth frame images are the first dynamic image of the first time, the sixth to sixteenth frame images are the first dynamic image of the second time, and the seventeenth to twenty-fourth frame images are the first dynamic image of the third time.

For example, it is assumed that, while the user is checking a live display of the first dynamic image of the first time, the user performs an operation to rotate the image at a time point of the third frame image.

In this case, the recognition processing section 420 recognizes the image rotation as the change processing in the first dynamic image of the first time.

In addition, for example, it is assumed that, while the user is checking a live display of the first dynamic image of the second time, the user performs an operation to perform gradation processing on the image at a time point of the ninth frame image.

In this case, the recognition processing section 420 recognizes the gradation processing as the change processing in the first dynamic image of the second time.

In addition, for example, it is assumed that, while the user is checking a live display of the first dynamic image of the third time, the user performs an operation to perform enhancement processing of enhancing the image at the twenty-first frame image.

In this case, the recognition processing section 420 recognizes the enhancement processing as the change processing in the first dynamic image of the third time.

The generation processing section 430 generates, based on the first dynamic image, a second dynamic image to be displayed on the display section 42 after the imaging by the radiation irradiation apparatus 30. The second dynamic image is constituted by a plurality of frame images corresponding to the plurality of frame images included in the first dynamic image, respectively.

The generation processing section 430 applies the change processing, which has been recognized by the recognition processing section 420, to the second dynamic image. Specifically, the change processing performed on the first dynamic image displayed live is applied to the second dynamic image. Note that, in addition to the change processing, correction processing and image processing suitable for the analysis of a dynamic image may also be applied to the second dynamic image.

For example, the generation processing section 430 generates a second dynamic image such that change processing is applied to the second dynamic image at a timing identical to a timing at which the change processing is applied to the first dynamic image. The information indicating the timing includes, for example, information indicating the frame image numbers and information indicating the number of frame images.

For example, in the first dynamic image of the first time in FIG. 6, the change processing related to the image rotation is applied at the time point of the third frame image, and thus, the change processing is also applied at the time point of the third frame image in the second dynamic image corresponding to the first dynamic image of the first time. That is, in the second dynamic image corresponding to the first dynamic image of the first time, the change processing related to the image rotation is not applied to the frame images before the third frame image, and the change processing related to the image rotation is applied to the third frame image and the subsequent frame images.

In addition, in the first dynamic image of the second time, the change processing related to the gradation processing is applied at the time point of the ninth frame image, and thus, the change processing is also applied at the time point of the ninth frame image of the second dynamic image corresponding to the first dynamic image of the second time. That is, in the second dynamic image corresponding to the first dynamic image of the second time, the change processing related to the gradation processing is not applied to the frame images before the ninth frame image, and the change processing related to the gradation processing is applied to the ninth frame image and the subsequent frame images.

In addition, in the first dynamic image of the third time, the change processing related to the enhancement processing is applied at the time of the 21st frame image, and thus, the change processing is also applied at the time point of the 21st frame image of the second dynamic image corresponding to the first dynamic image of the third time. That is, in the second dynamic image corresponding to the first dynamic image of the third time, the change processing related to the enhancement processing is not applied to the frame images before the 21st frame image, and the change processing related to the enhancement processing is applied to the 21st frame image and the subsequent frame images.

In this manner, the aspect of the first dynamic image during live display is reproduced when the second dynamic image is displayed on the display section 42.

The storage processing section 440 stores a first dynamic image and a second dynamic image in the storage section 43. In addition, the storage processing section 440 stores a first dynamic image in association with the content of the change processing in the storage section 43 for each frame image.

In addition, the recognition processing section 420 may recognize the change processing based on the content of the change processing associated with each frame image.

Incidentally, in a live display, frame images to be displayed may be decimated in order to ensure real-time capability depending on the performance of an apparatus, a network, or the like. For example, it is assumed that, when the first dynamic image of the first time is displayed live, only the first, third, and fifth frame images are displayed due to decimation. This case means that the second and fourth frame images have not subjected to change processing, whereas the first, third, and fifth frame images have been associated with the change processing. For this reason, the recognition processing section 420 may recognize the change processing on the second and fourth frame images based on the content of the change processing associated with the first, third, and fifth frame images. Thus, a live display in a non-decimated state can be reproduced.

In addition, the display processing section 410 displays the second dynamic image after the display of the first dynamic image ends, that is, after the imaging by the radiation irradiation apparatus 30 is completed. In this case, the display processing section 410 replaces the display of the first dynamic image on the display section 42 with the display of the second dynamic image.

In addition, in the intermittent imaging mode, the display processing section 410 may replace, during an interruption period between captured images, the display of the first dynamic image immediately before the interruption with the display of the second dynamic image corresponding to the first dynamic image.

In this case, the frame image corresponding to the final frame image of the first dynamic image immediately before the interruption is displayed. This frame image is displayed, on the display section 42, in a state in which the change processing based on the first dynamic image has been applied to the second dynamic image.

For example, in the example illustrated in FIG. 6, no radiographic dynamic image has been captured during the period between the first dynamic image of the first time and the first dynamic image of the second time. At the time of the end of the imaging of the first dynamic image of the first time, the fifth frame image, which is the final frame image of the first dynamic image of the first time, is displayed on the display section 42. For this reason, during the period between the first dynamic image of the first time and the first dynamic image of the second time, the display of the fifth frame image in the first dynamic image is replaced, once the generation of the second dynamic image is completed, with the display of the fifth frame image in the second dynamic image.

In addition, in this case, the second dynamic image may be played back and displayed when the user presses a playback button.

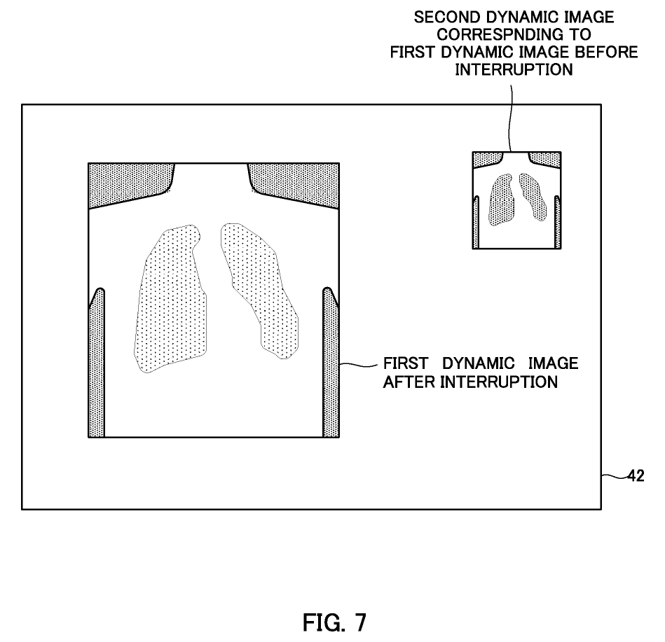

In addition, when the first dynamic image after an interruption is displayed on the display section 42 in the intermittent imaging mode, the display processing section 410 may display, at a position different from the position of the first dynamic image on the display section 42, the second dynamic image corresponding to the first dynamic image before the interruption (for example, see FIG. 7).

In addition, the display processing section 410 may switch between the second dynamic image and a dynamic image to which change processing has not been applied and display the switched image on the display section 42, based on operation information on the operation of the user operation section 41.

The dynamic image to which change processing has not been applied may be, for example, the original image of the first dynamic image, or may be a dynamic image to which various types of corrections have been applied to the original image such that dynamic analysis can be performed.

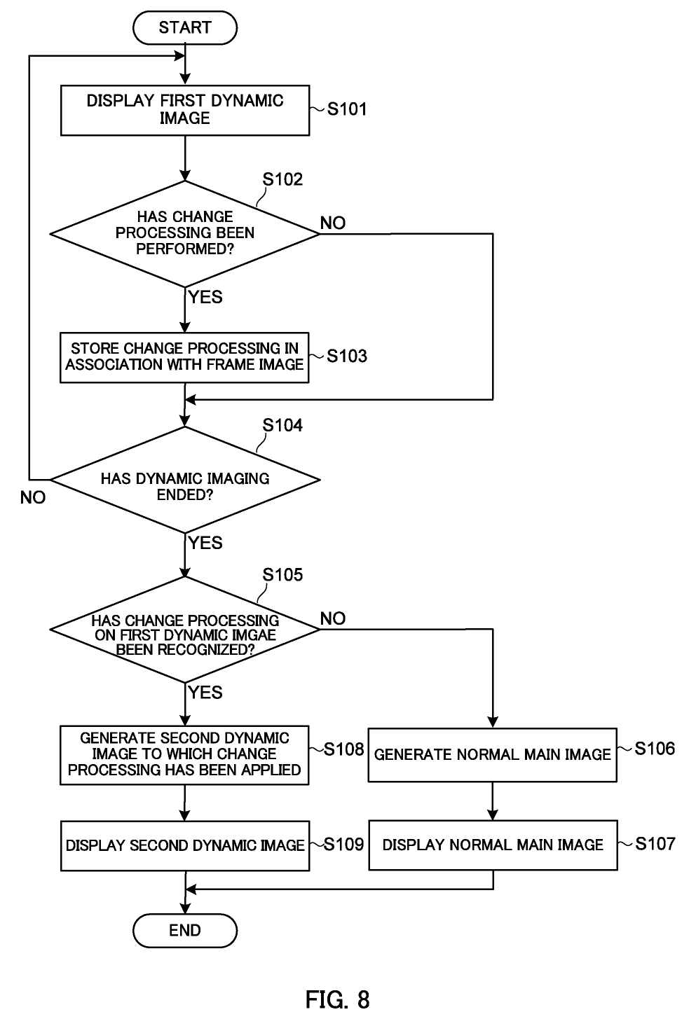

Next, a flow of processing performed by the imaging control section 40 will be described. FIG. 8 is a flowchart illustrating exemplary operations of processing performed by the imaging control section 40. This control is executed as appropriate in the radiographic imaging system 1 when an instruction to start dynamic imaging is acquired.

As illustrated in FIG. 8, the imaging control section 40 displays a first dynamic image on the display section 42 (step S101). After step S101, the imaging control section 40 determines whether change processing has been performed on the first dynamic image (step S102).

In a case where it has been determined that no change processing has been performed (step S102, NO), the processing proceeds to step S104. In a case where change processing has been performed (step S102, YES), on the other hand, the imaging control section 40 stores the change processing in association with the frame image in the storage section 43 (step S103).

After step S103, the imaging control section 40 determines whether the dynamic imaging has ended (step S104). In a case where it has been determined that the dynamic imaging has not ended (step S104, NO), the processing returns to step S101. In a case where the dynamic imaging has ended (step S104, YES), on the other hand, the imaging control section 40 determines whether the change processing on the first dynamic image stored in the storage section 43 has been recognized (step S105).

In a case where it has been determined that the change processing has not been recognized (step S105, NO), the imaging control section 40 generates a normal main image, which is a dynamic image to which no change processing has been applied (step S106). After step S106, the imaging control section 40 causes the display section 42 to display the normal main image (step S107).

In a case where it has been determined that the change processing has been recognized (step S105, YES), on the other hand, the imaging control section 40 generates a second dynamic image to which the change processing has been applied (step S108). After step S108, the imaging control section 40 causes the display section 42 to display the second dynamic image (step S109). After step S107 or step S109, the present control ends.

Note that, the processing in steps S105, S106, and S108 may be performed concurrently during dynamic imaging.

According to the present embodiment configured as described above, a second dynamic image is generated by applying change processing on a first dynamic image to the second dynamic image. As a result, when checking the main image (the second dynamic image), it is no longer necessary for the user to perform change processing on the second dynamic image, and thus, it is possible to reduce the labor of the user in checking the second dynamic image. That is, in the present embodiment, convenience in the radiographic imaging system 1 capable of live display can be improved.

In addition, since processing performed on the first dynamic image based on operation information on the operation of the user is recognized as change processing, a second dynamic image to which change processing in line with the intention of the user has been applied can be generated.

In addition, since change processing is applied to a second dynamic image at a timing identical to a timing of the change processing on a first dynamic image, the first dynamic image displayed live can be reproduced when checking the second dynamic image.

Note that, in the above-described embodiment, a second dynamic image is generated such that change processing is applied to the second dynamic image at a timing identical to a timing at which the change processing is applied to the first dynamic image, but the present invention is not limited thereto. For example, the generation processing section 430 may apply, to every frame image of a second dynamic image, processing performed on a final frame image among a plurality of frame images included in a first dynamic image.

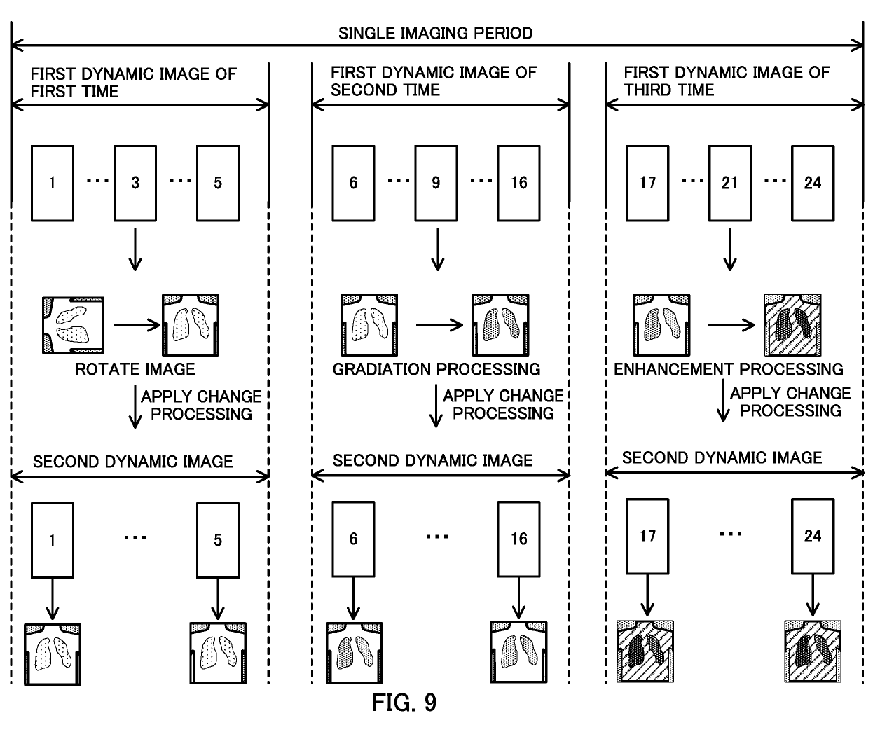

For example, in the first dynamic image of the first time in FIG. 9, the change processing related to the image rotation is applied at the time point of the third frame image, and thus, the change processing related to the image rotation is applied to the fifth frame image, which is the final frame image. For this reason, the generation processing section 430 applies the change processing related to the image rotation to every frame image of the second dynamic image.

In addition, in the first dynamic image of the second time, the change processing related to the gradation processing is applied at the time point of the ninth frame image, and thus, the change processing related to the gradation processing is applied to the sixteenth frame image, which is the final frame image. For this reason, the generation processing section 430 applies the change processing related to the gradation processing of the image to every frame image of the second dynamic image.

In addition, in the first dynamic image of the third time, the change processing related to the enhancement processing is applied at the time point of the twenty-first frame image, and thus, the change processing related to the enhancement processing is applied to the twenty-fourth frame image, which is the final frame image. For this reason, the generation processing section 430 applies the change processing related to the enhancement processing to every frame image of the second dynamic image.

In addition, in a case where some kind of change processing has been performed on an initial frame image among a plurality of frame images included in a first dynamic image, or the like, the generation processing section 430 may apply the change processing performed on the initial frame image to a second dynamic image, even when another change processing is performed thereafter.

For example, it is assumed that, during an interruption period after imaging of a first dynamic image of the first time is completed, the orientation of the FPD 20 is changed in consideration of the body shape of the subject. Along therewith, the subject is rotated in advance as an imaging condition prior to resuming the imaging on the assumption that the image will be rotated, so that the subject is displayed in the same orientation on the display. Although such a case means that change processing has been performed in the imaging control section 40, no change processing has been performed on the display. In such a case, the generation processing section 430 applies the change processing performed on the initial frame image to the second dynamic image.

In addition, the recognition processing section 420 may recognize, as change processing, processing applied to a frame image designated by the user among a plurality of frame images constituting a first dynamic image. In this case, the generation processing section 430 may apply, to every frame image of a second dynamic image corresponding to the first dynamic image, change processing related to the frame image designated by the user.

In addition, in the above-described embodiment, the change processing is recognized based on a single operation of the user. However, there is a case where the operation information on the operation of the user includes operations of the user performed a plurality of times on one first dynamic image. In this case, the recognition processing section 420 may recognize, as change processing, the final processing based on the operation of the user performed last among the operations of the user performed the plurality of times.

For example, in a case where a plurality of types of gradation processing has been performed on one first dynamic image, the gradation processing performed last is recognized as change processing. Note that, the recognition processing section 420 may recognize the final processing as change processing in a case where it can be determined that the final processing is optimal among pieces of change processing based on operations performed a plurality of times.

In addition, the recognition processing section 420 may recognize, according to the content of the final processing based on the operation performed last among operations performed a plurality of times, processing other than the final processing as the change processing.

For example, it is assumed that gradation processing and enhancement processing have been performed in sequence on one first dynamic image. The enhancement processing is performed for a predetermined reason, such as for identifying a structure such as the leading end of a catheter, for example. For this reason, for example, in a case where a first dynamic image includes a frame image for confirming a matter other than the insertion of a structure, it is not necessary to apply the enhancement processing to every frame image of the main image.

For this reason, the recognition processing section 420 recognizes, as change processing, the gradation processing other than the enhancement processing which is the final processing.

In addition, in the above-described embodiment, in a case where captured images include a plurality of dynamic images in the intermittent imaging mode, second dynamic images are generated for the plurality of dynamic images, respectively, but the present invention is not limited thereto. For example, a second dynamic image may also be generated for every frame image in a single imaging period in the intermittent imaging mode.

In addition, in the above-described embodiment, change processing set in advance is applied, but the present invention is not limited thereto. For example, the generation processing section 430 may change, based on information on the imaging purpose of a first dynamic image, the method of applying change processing to a second dynamic image.

The information on the imaging purpose is, for example, information on the purpose for capturing a radiographic image, such as confirming the insertion of a catheter and the evaluation of swallowing, and is determined based on, for example, examination order information, information on the medical department that has requested the imaging, and the like.

In a case where it is determined based on information on an examination order, the generation processing section 430 determines information on the imaging purpose, for example, by extracting information of the imaging purpose described in the examination order. In addition, in a case where it is determined based on information on the medical department that has requested the imaging, the generation processing section 430 determines information on the imaging purpose, for example, by referring to a table or the like in which information on the imaging purpose associated with information on the medical department is described.

For example, the generation processing section 430 selects a method of generating a second dynamic image by referring to a table in which information on the imaging purpose is associated with change processing.

For example, it is assumed that the generation processing section 430 determines, based on information on an examination order or information on a medical department, that the imaging purpose is to confirm the insertion of a catheter.

In this case, the generation processing section 430 generates a second dynamic image such that change processing is applied to the second dynamic image at a timing identical to a timing at which the change processing is applied to the first dynamic image.

In addition, it is assumed that the generation processing section 430 determines, based on information on an examination order or information on a medical department, that the imaging purpose is to evaluate swallowing.

In this case, the generation processing section 430 generates a second dynamic image such that processing performed on the final frame image among a plurality of frame images included in a first dynamic image is applied to every frame image of the second dynamic image.

As described above, since the method of applying change processing can be changed depending on the imaging purpose, appropriate change processing can be performed on a second dynamic image in line with the imaging purpose.

In addition, in the above-described embodiment, a dynamic image subjected to the same change processing as that applied to the first dynamic image displayed live is generated as a second dynamic image, but the present invention is not limited thereto. For example, the generation processing section 430 may apply, as change processing, more detailed processing than the processing when a live display is performed, to the second dynamic image.

Since the first dynamic image needs to be displayed on the display section 42 in real time when change processing related to image processing such as gradation processing is performed, relatively simple processing is performed on the radiographic dynamic image acquired from the radiation irradiation apparatus 30. However, since the second dynamic image needs not be displayed in real time, it is considered in the case of change processing related to image processing that relatively detailed processing is preferably performed from the viewpoint of user confirmation.

For this reason, an image can be easier for the user to confirm by performing, on a second dynamic image, more detailed processing than the processing performed on a first dynamic image.

In addition, in the above-described embodiment, change processing performed on a first dynamic image by an operation of the user during live display is applied to a second dynamic image, but the present invention is not limited thereto. For example, the recognition processing section 420 may recognize the content of change processing based on an analysis result of a first dynamic image displayed during live display. The image analysis may be performed by the imaging control section 40, or may be performed by an external apparatus such as the dynamic analysis apparatus 5.

For example, it is assumed that the imaging purpose is to confirm the insertion of a catheter. In this case, image analysis makes it possible to identify, in a dynamic image, the timing at which the leading end portion of the catheter is inserted into the subject. In this case, the recognition processing section 420 recognizes change processing to be applied to images from the frame image at that timing onward, for example, as enhancement processing or the like.

In addition, in a case where the imaging purpose is to evaluate swallowing, image analysis makes it possible to identify, in a dynamic image, the timing at which barium enters the body of the subject. In this case, the recognition processing section 420 recognizes change processing to be applied to images from the frame image at that timing onward, for example, as enhancement processing or the like.

In this way, processing that is considered to be performed when the user checks the main image can be performed in advance of the user, and thus, convenience in checking a second dynamic image can be improved.

In addition, in the above-described embodiment, the imaging control section 40 is exemplified as the radiographic image processing apparatus, but the present invention is not limited thereto. For example, the radiographic image processing apparatus may be another apparatus such as the RIS 4.

In addition, in the above-described embodiment, a display medium (the display section 42) is provided in the radiographic imaging system 1, but the present invention is not limited thereto, and the radiographic imaging system may not be provided with any display medium. In this case, for example, an output apparatus in the imaging room, the PACS 2, the HIS 3, the RIS 4, or the like may include a display medium.

In addition, any of the embodiment described above is only illustration of an exemplary embodiment for carrying out the present invention, and the technical scope of the present invention shall not be construed limitedly thereby. That is, the present invention can be carried out in various forms without departing from the gist or the main features thereof.

Although embodiments of the present invention have been described and illustrated in detail, the disclosed embodiments are made for purposes of illustration and example only and not limitation. The scope of the present invention should be interpreted by terms of the appended claims.

What is claimed is:

1. A radiographic image processing apparatus, comprising
a hardware processor that displays, on a display medium during imaging by a radiographic imaging apparatus, a first dynamic image of a subject irradiated with radiation, wherein
the hardware processor
recognizes change processing on at least one of a plurality of frame images included in the first dynamic image,
generates, based on the first dynamic image, a second dynamic image to be displayed on the display medium after the imaging by the radiographic imaging apparatus, and
applies the change processing to the second dynamic image.

2. The radiographic image processing apparatus according to claim 1, wherein
the hardware processor recognizes, as the change processing, processing performed on the first dynamic image displayed on the display medium based on operation information on the operation of the user.

3. The radiographic image processing apparatus according to claim 2, wherein
the hardware processor generates the second dynamic image such that the change processing is applied to the second dynamic image at a timing identical to a timing at which the change processing is applied to the first dynamic image.

4. The radiographic image processing apparatus according to claim 2, wherein
the hardware processor applies, to every frame image of the second dynamic image, processing performed on a final frame image among the plurality of frame images included in the first dynamic image.

5. The radiographic image processing apparatus according to claim 2, wherein
the hardware processor
recognizes, as the change processing, processing applied to a frame image designated by a user among the plurality of frame images constituting the first dynamic image, and
applies, to every frame image of the second dynamic image, change processing related to the processing applied to the frame image designated by the user.

6. The radiographic image processing apparatus according to claim 2, wherein
the hardware processor applies, as the change processing, more detailed processing than the processing to the second dynamic image.

7. The radiographic image processing apparatus according to claim 1, wherein
the hardware processor changes, based on information on an imaging purpose of the first dynamic image, a method of applying the change processing to the second dynamic image.

8. The radiographic image processing apparatus according to claim 7, wherein
the hardware processor determines a content of the imaging purpose based on examination order information or medical department information.

9. The radiographic image processing apparatus according to claim 1, wherein
the hardware processor recognizes the change processing based on an image analysis result of the first dynamic image.

10. The radiographic image processing apparatus according to claim 1, wherein:
the radiographic imaging apparatus is capable of performing imaging in an intermittent imaging mode which allows a dynamic image or a still image to be captured a plurality of times, the dynamic image or the still image having been captured during an imaging period from an imaging start to an imaging end,
the first dynamic image is captured images captured the plurality of times during the imaging period in the intermittent imaging mode, and
the captured images captured the plurality of times include one or more dynamic images.

11. The radiographic image processing apparatus according to claim 1, wherein
the change processing is processing for changing at least one of adjustment of a region of interest, setting of an effective image region, adjustment of an image size, image rotation, image inversion, adjustment of an S/G value, image black-and-white inversion, predetermined image processing, enhancement processing, and/or grid pattern removal processing.

12. The radiographic image processing apparatus according to claim 1, wherein
the hardware processor stores, in a storage apparatus, the first dynamic image in association with the change processing for each of the plurality of frame images.

13. A generation method in a radiographic image processing apparatus, the generation method comprising:
displaying, on a display medium during imaging by a radiographic imaging apparatus, a first dynamic image of a subject irradiated with radiation;
recognizing change processing on at least one of a plurality of frame images included in the first dynamic image; and
generating, based on the first dynamic image, a second dynamic image to be displayed on the display medium after the imaging by the radiographic imaging apparatus, wherein
generating the second dynamic image includes applying the change processing to the second dynamic image.

14. A radiographic image system, comprising:
a radiation irradiation apparatus capable of capturing a radiographic dynamic image; and
the radiographic image processing apparatus according to claim 1.
